(19) United States
(12) Patent Application Publication
(10) Pub. No.: US 2015/0045391 A1
(43) Pub. Date: Feb. 12, 2015

(54) FILANESIB COMBINED WITH POMALIDOMIDE DISPLAYS ENHANCED ANTI-TUMOR ACTIVITY

(71) Applicant: Array BioPharma Inc., Boulder, CO (US)

(72) Inventors: Deborah A. Anderson, Boulder, CO (US); Michael J. Humphries, Boulder, CO (US); Robert A. Rieger, Boulder, CO (US); Lance M. Williams, Boulder, CO (US)

(21) Appl. No.: 14/454,626

(22) Filed: Aug. 7, 2014

Related U.S. Application Data
(60) Provisional application No. 61/863,815, filed on Aug. 8, 2013.
(60) Provisional application No. 61/909,871, filed on Nov. 27, 2013.

(51) Int. Cl. A61K 31/454; A61K 31/433

(52) U.S. Cl. CPC A61K 31/454; A61K 31/433
USPC 514/323

(57) ABSTRACT

A combination of filanesib and pomalidomide for treating patients is provided.

### RELATED APPLICATIONS

This application claims priority to U.S. Provisional Application No. 61/863,815 that was filed on Aug. 8, 2013 and U.S. Provisional Application No. 61/909,871 that was filed on Nov. 27, 2013. The entire content of these provisional applications are hereby incorporated herein by reference.

### BACKGROUND OF THE INVENTION

1. Field of the Invention

The present invention relates to the combination of filanesib and pomalidomide in treating patients.

2. Description of the State of the Art

Filanesib, (S)-2-(3-aminopropyl)-5-(2,5-difluorophenyl)-N-methoxy-N-methyl-2-phenyl-1,3,4-thiadiazole-3(2H)-carboxamide hydrochloride, also known as “ARRY-520”, which has the structure:

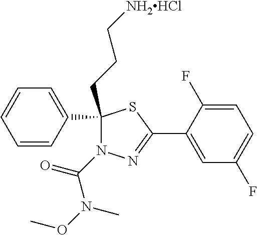

is a kinesin spindle protein (“KSP”) inhibitor (see U.S. Pat. No. 7,449,486, US 2010/0099697, WO 2010/045624 and PCT/US2013/54807, the contents of which are herein incorporated by reference in their entirety). KSP inhibition results in mitotic arrest of proliferating cells and subsequent cell death. Filanesib has shown clinical activity in patients with relapsed and refractory multiple myeloma (“MM”).

Inhibitors of KSP, a mitosis-specific motor protein, represent a novel class of targeted anti-cancer therapies that have demonstrated clinical activity in hematological cancers. Inhibition of KSP results in the formation of aberrant monopolar spindles, mitotic arrest, and rapid apoptosis through degradation of the survival protein Mc1-1. The majority of KSP-inhibitor sensitive cells are proliferating hematopoietic cells which are dependent on Mc1-1 for survival. Filanesib, a highly selective KSP inhibitor, has been in Phase 2 clinical studies in patients with relapsed and refractory multiple myeloma. To date, filanesib has demonstrated a well-tolerated safety profile and clinical activity both alone and in combination with bortezomib, dexamethasone, or carfilzomib in heavily pretreated patients. While prior preclinical studies have shown that filanesib is additive or synergistic when combined with bortezomib in several in vivo models of multiple myeloma, the combinability of filanesib with other myeloma standards of care, such as immunomodulatory drugs, has not been thoroughly investigated.

Pomalidomide (Pomalyst®), an immunomodulatory drug approved by the FDA, is a thalidomide analogue indicated for patients with multiple myeloma who have received at least two prior therapies including lenalidomide and bortezomib and have demonstrated disease progression on or within 60 days of completion of the last therapy. Approval was based on response rate. Clinical benefit, such as improvement in survival or symptoms, has not been verified. The recommended starting dose of pomalidomide is 4 mg once daily orally on days 1-21 of repeated 28-day cycles until disease progression. Pomalidomide may be given in combination with dexamethasone. The dose of pomalidomide may be modified for hematological toxicities, generally starting at 1 mg less than the previous dose (i.e., 3 mg, 2 mg or 1 mg) until discontinuation of pomalidomide (see pomalidomide prescribing information for more information).

### SUMMARY OF THE INVENTION

Surprisingly, it has been found that the combination of filanesib and pomalidomide is a well-tolerated and synergistic in vitro and in vivo regimen.

In one aspect, the present invention relates to treating cancer comprising administering to a mammal in need of such treatment an effective amount of filanesib and pomalidomide.

In another aspect, treating cancer in a mammal comprising administering a therapeutically effective amount of filanesib and pomalidomide to the mammal is provided.

In a further aspect, the cancer is a hematological tumor. In a further aspect, the cancer is selected from lymphomas, leukemia and multiple myeloma.

In another aspect, the cancer is selected from solid tumors. In a further aspect, the cancer is selected from skin, breast, brain, cervical carcinoma, and testicular cancer.

In another aspect, the cancer is selected from solid tumors. In another aspect, the cancer is selected from breast cancer, colorectal cancer, non-small cell lung cancer, pancreatic cancer, bladder cancer, salivary gland cancer (adenoid cystic), esophageal cancer, mesothelioma cancer, and mixed small cell lung cancer/non-small cell lung cancer.

### BRIEF DESCRIPTION OF THE FIGURES

FIG. 1 shows the mean plasma concentration of filanesib and/or pomalidomide.

FIG. 2 shows a tumor growth inhibition (“TGI”) experiment in mice with H929 xenografts.

FIG. 3 shows a TGI experiment in mice with RPMI-8226 xenografts.

FIG. 4 shows a TGI experiment in mice with JJN3 xenografts.

FIG. 5 shows the mean platelet count in mice.

FIG. 6 shows the mean lymphocyte count in mice.

FIG. 7 shows the mean neutrophil count in mice.

FIG. 8 shows the percent body weight changes in a TGI experiment in mice with RPMI-8226 xenografts.

FIG. 9 shows a TGI experiment in mice with RPMI-8226 xenografts.

### DETAILED DESCRIPTION OF THE INVENTION

Reference will now be made in detail to certain embodiments. While enumerated embodiments will be described, it will be understood that they are not intended to limit the invention to those embodiments. On the contrary, the invention is intended to cover all alternatives, modifications, and equivalents, which may be included within the scope of the present invention as defined by the claims. One skilled in the art will recognize many methods and materials similar or equivalent to those described herein, which could be used in the practice of the present invention. The present invention is in no way limited to the methods and materials described. In the event that one or more of the incorporated literature and similar materials differs from or contradicts this application, including but not limited to defined terms, term usage, described techniques, or the like, this application controls.

### Definitions

Certain embodiments of this invention encompass methods of treating, preventing and/or managing various types of cancer and diseases and associated disorders. As used herein, unless otherwise specified, the term “treating” or “treat” refers to the administration of filanesib and pomalidomide or other additional active agent after the onset of symptoms of the particular disease or disorder. Beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” may also mean prolonging survival as compared to expected survival if not receiving treatment. Those in need of treatment include those already with the condition or disorder, as well as those prone to have the condition or disorder. As used herein, unless otherwise specified, the term “preventing” refers to the administration prior to the onset of symptoms, particularly to patients at risk of cancer, and other diseases and associated disorders. The terms “treat” or “treatment” may also refer to therapeutic or palliative measures. The term “prevention” includes the inhibition of a symptom of the particular disease or disorder. Patients with familial history of cancer and diseases and associated disorders are preferred candidates for preventive regimens. As used herein and unless otherwise indicated, the term “managing” encompasses preventing the recurrence of the particular disease or disorder in a patient who had suffered from it, and/or lengthening the time a patient who had suffered from the disease or disorder remains in remission.

The term “about” is used herein to mean approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 20%.

The terms “cancer” and “cancerous” refer to or describe the physiological condition in mammals that is typically characterized by abnormal or unregulated cell growth. A “tumor” comprises one or more cancerous cells. Examples of cancer include, but are not limited to, carcinoma, lymphoma, blastoma, sarcoma, and leukemia or lymphoid malignancies. More particular examples of such cancers include squamous cell cancer (e.g., epithelial squamous cell cancer), lung cancer including small cell lung cancer, non-small cell lung cancer (“NSCLC”), adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, skin cancer, including melanoma, as well as head and neck cancer.

The phrase “pharmaceutically acceptable” indicates that the substance or composition is compatible chemically and/or toxicologically, with the other ingredients comprising a formulation, and/or the mammal being treated therewith.

The phrase “pharmaceutically acceptable salt,” as used herein, refers to pharmaceutically acceptable organic or inorganic salts of a compound described herein.

The phrases “therapeutically effective amount” or “effective amount” mean an amount of a compound described herein that, when administered to a mammal in need of such treatment, sufficient to (i) treat or prevent the particular disease, condition, or disorder, (ii) attenuate, ameliorate, or eliminate one or more symptoms of the particular disease, condition, or disorder, or (iii) prevent or delay the onset of one or more symptoms of the particular disease, condition, or disorder described herein. The amount of a compound that will correspond to such an amount will vary depending upon factors such as the particular compound, disease condition and its severity, the identity (e.g., weight) of the mammal in need of treatment, but can nevertheless be routinely determined by one skilled in the art.

The term “mammal” means a warm-blooded animal that has or is at risk of developing a disease described herein and includes, but is not limited to, guinea pigs, dogs, cats, rats, mice, hamsters, and primates, including humans.

Combination of Filanesib and Pomalidomide

The combination utility of filansib and pomalidomide has been evaluated in preclinical models of multiple myeloma. Our findings show that the combination of filanesib (12.5 mg/kg, IP, D1, 2) with pomalidomide (10 mg/kg, IP, QD) is significantly more active than either monotherapy alone in several in vivo models of multiple myeloma. In particular, complete responses and cures were observed in two models. In the RPMI-8226 model, a 100% cure rate was observed with the combination regimen as compared to a 0% cure rate for either monotherapy (cure is defined as no palpable tumor 100 days from study initiation). Likewise, in the JJN3 model a 100% complete response rate was observed with the combination regimen versus a 0% complete response rate for either monotherapy (complete response is defined as no palpable tumor for 2 consecutive tumor measurements). Notably, similar combination efficacy was also observed in the RPMI-8226 model at full and significantly reduced doses of both filanesib and pomalidomide, supporting a synergistic interaction between these drugs. The combination regimen was well tolerated causing no significant enhancement of body weight loss (not exceeding 10% for any group). Additionally, hematology analysis showed that the combination regimen caused no significant increase in thrombocytopenia or leukopenia. Consistent with clinical findings, both single agent filanesib and pomalidomide caused neutropenia while the combination of these drugs moderately enhanced these effects. Pharmacokinetic studies confirmed that administration of pomalidomide with filanesib does not alter the exposure of either compound, suggesting that the mechanism of enhanced anti-tumor activity is dependent upon pharmacodynamic rather than pharmacokinetic effects.

Filanesib is typically administered intravenously. Filanesib is generally provided as a lyophilized powder contained in a Type 1 clear glass vial for IV use. The powder is reconstituted with sterile water for injection to form a solution and diluted with normal saline prior to IV administration.

The major dose limiting toxicity (“DLT”) of filanesib has been found to be neutropenia. As such, prophylactic granulocyte colony-stimulating factory (“G-CSF”) may be administered.

Filanesib is generally administered on Days 1 and 2 of a 14 day cycle (Days 1 and 2 Q2W). Filanesib is generally administered on this schedule at 2.5 mg/m²/cycle (1.25 mg/m²/day) without G-CSF and 3.0 mg/m²/cycle (1.5 mg/m²/day) with prophylactic G-CSF. However, filanesib may also be administered on Day 1 of a 14 day cycle (Day 1 Q2W) or Day 1 and 15 on a 28 day cycle (Days 1 and 15 Q4W). Filanesib may also be administered at 2.5 mg/m²/cycle (1.25 mg/m²/day) with G-CSF.

The predicted in vitro IC₅₀ of filanesib is about 0.2 ng/mL. In a further embodiment, the predicted in vitro IC₅₀ of filanesib is 0.2 ng/mL.

In certain embodiments, the cancer is selected from breast, ovary, cervix, prostate, testis, genitourinary tract, esophagus, larynx, glioblastoma, neuroblastoma, stomach, skin, keratoacanthoma, lung, epidermoid carcinoma, large cell carcinoma, NSCLC, small cell carcinoma, lung adenocarcinoma, bone, colon, adenoma, pancreas, adenocarcinoma, thyroid, follicular carcinoma, undifferentiated carcinoma, papillary carcinoma, seminoma, melanoma, sarcoma, bladder carcinoma, liver carcinoma and biliary passages, kidney carcinoma, myeloid disorders, lymphoid disorders, hairy cells, buccal cavity and pharynx (oral), lip, tongue, mouth, pharynx, small intestine, colon-rectum, large intestine, rectum, brain and central nervous system, Hodgkin's and leukemia.

In certain embodiments, the cancer is a hematological cancer. In certain embodiments, the cancer is selected from lymphomas, leukemia and multiple myeloma. In certain embodiments, the cancer is selected from leukemia and multiple myeloma. In certain embodiments, the cancer is selected from acute myeloid leukemia and multiple myeloma. In certain embodiments, the cancer is multiple myeloma. In certain embodiments, the cancer is acute myeloid leukemia.

In certain embodiments, the cancer is a solid tumor. In certain embodiments, the cancer is selected from skin, breast, brain, cervical carcinoma, and testicular cancer. In certain embodiments, the cancer is selected from breast cancer, colorectal cancer, non-small cell lung cancer, pancreatic cancer, bladder cancer, salivary gland cancer (adenoid cystic), esophageal cancer, mesothelioma cancer, and mixed small cell lung cancer/non-small cell lung cancer.

Co-administration of filanesib with pomalidomide substantially enhanced efficacy. Marked increases in complete response rates were observed in two of three MM models. Dose reductions of both compounds displayed equivalent combination efficacy.

Co-administration of filanesib with pomalidomide did not alter exposure.

Co-administration of filanesib with pomalidomide displayed acceptable tolerability. There were no marked decreases in lymphocytes or platelets, and body weight loss was minor and acceptable. Neutrophil decreases were expected, co-administration moderately enhanced neutrophil loss (day 5) that was rapidly reversible (day 12).

Additional Combination Therapy

The compounds described herein and stereoisomers and pharmaceutically acceptable salts thereof may be employed alone or in combination with other therapeutic agents for treatment. The compounds described herein may be used in combination with one or more additional drugs, for example an anti-hyperproliferative (or anti-cancer) agent that works through action on a different target protein. The second compound of the pharmaceutical combination formulation or dosing regimen preferably has complementary activities to the compound described herein, such that they do not adversely affect each other. Such molecules are suitably present in combination in amounts that are effective for the purpose intended. The compounds may be administered together in a unitary pharmaceutical composition or separately and, when administered separately this may occur simultaneously or sequentially in any order. Such sequential administration may be close in time or remote in time.

In certain embodiments, G-CSF is administered in combination with filanesib and pomalidomide.

In certain embodiments, dexamethasone is administered in combination with filanesib. In certain embodiments, dexamethasone is administered in combination with filanesib and pomalidomide. In certain embodiments, G-CSF is administered in combination with filanesib and dexamethasone. In certain embodiments, G-CSF is administered in combination with filanesib, pomalidomide and dexamethasone.

### EXAMPLES

For illustrative purposes, the following Examples are included. However, it is to be understood that these Examples do not limit the invention and are only meant to suggest a method of practicing the invention.

In the Examples described below, unless otherwise indicated all temperatures are set forth in degrees Celsius. Reagents were purchased from commercial suppliers, and were used without further purification unless otherwise indicated.

### Example 1

### Co-Administration of Filanesib and Pomalidomide Does Not Impact Exposure

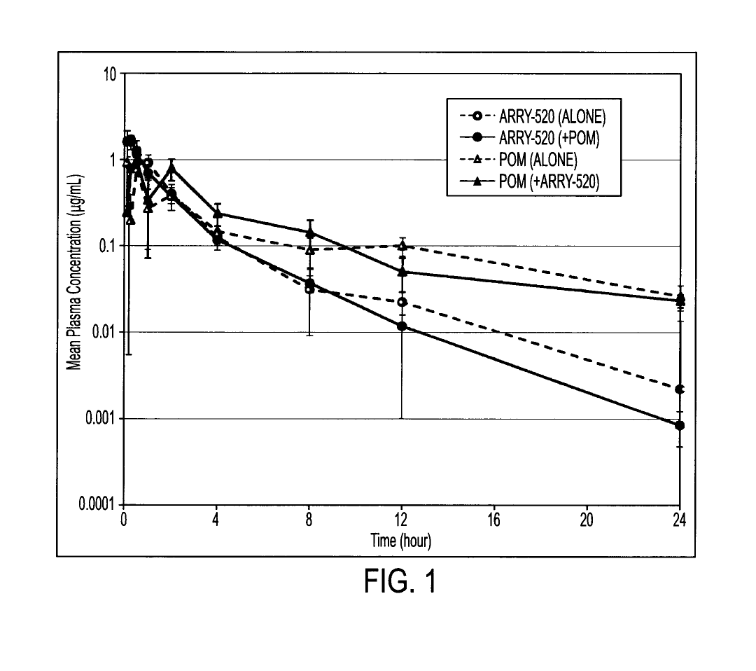

Naïve male CD-1 mice were administered a single intraperitoneal (“IP”) bolus dose of filanesib (12.5 mg/kg) and/or pomalidomide (10 mg/kg). Blood was collected at 4, 8, 12, 16, 20 and 24 hours via cardiac puncture, ethylenediaminetetraacetic acid (“EDTA”) plasma was prepared, and concentration of plasma analytes were measured. See FIG. 1 and Table 1.

TABLE 1

| | Cₘₐₓ | AUCₗₐₛₜ | Tₘₐₓ |
|---|---|---|---|
| Regimen | (μg/mL) | (μg*hour/mL) | (hour) |
| filanesib (alone) | 1.66 | 3.09 | 0.083 |
| filanesib + pomalidomide | 1.76 | 2.77 | 0.25 |
| pomalidomide (alone) | 0.956 | 3.08 | 0.083 |
| pomalidomide + filanesib | 0.997 | 3.95 | 0.50 |

### Example 2

### Xenograft

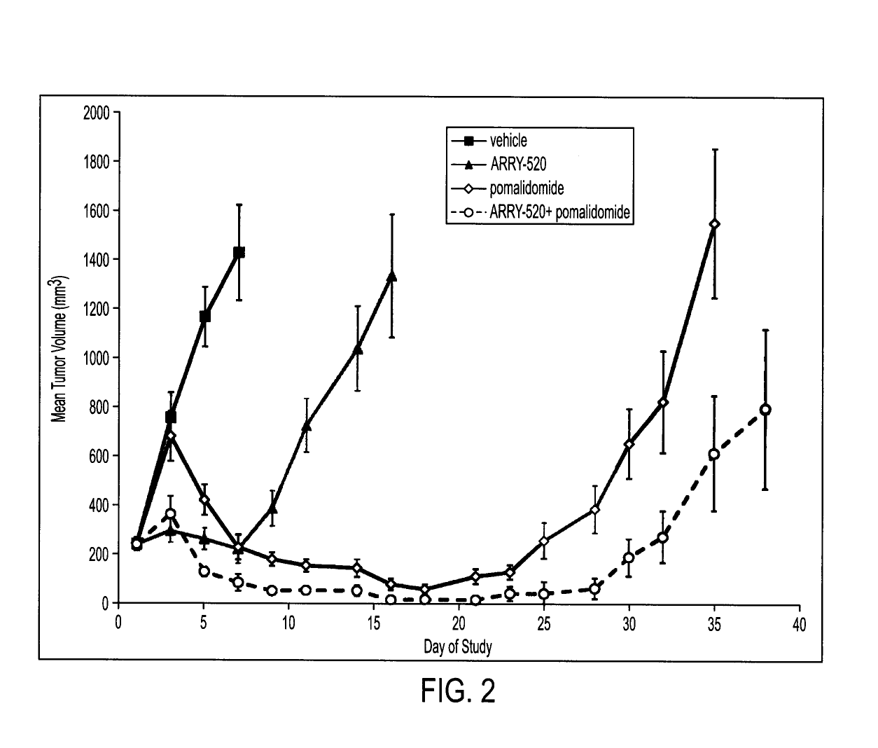

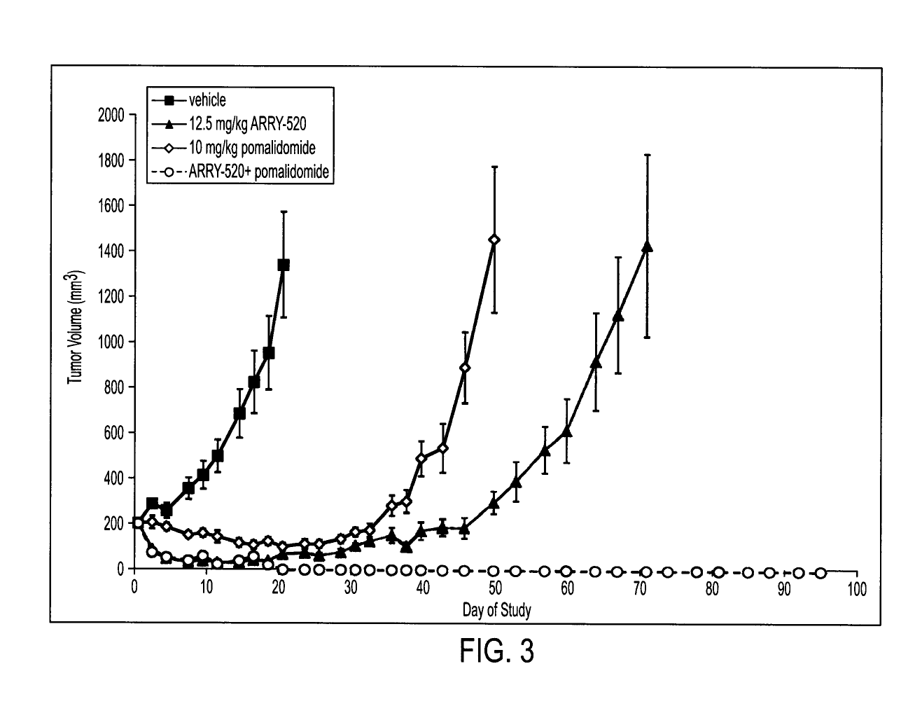

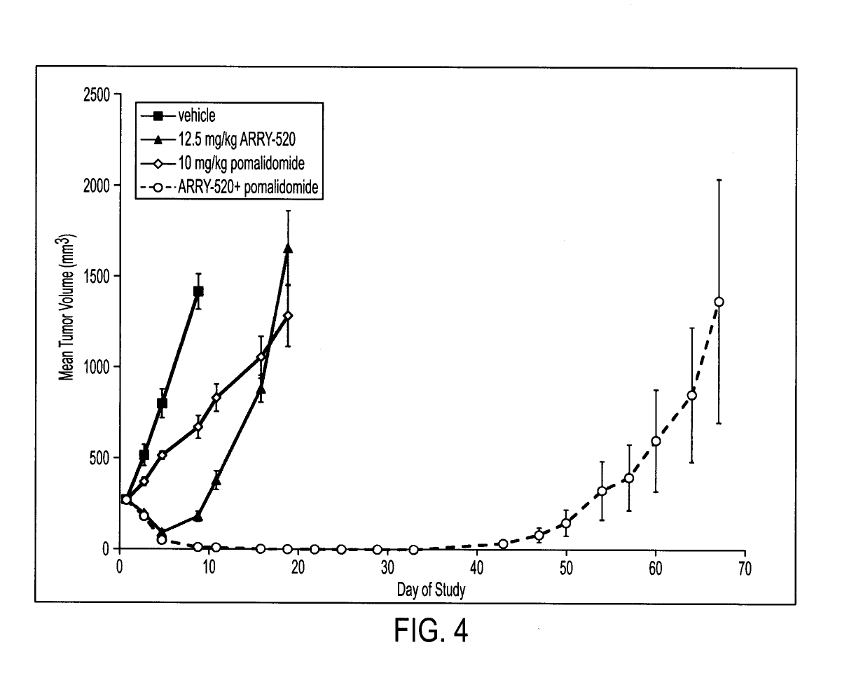

Female SCID-beige mice were inoculated subcutaneously with 10×10⁶ H929 (FIG. 2), RPMI-8226 (FIG. 3) or JJN3 (FIG. 4) tumor cells in 50% 1× phosphate buffered saline (“PBS”) 50% Matrigel™ (100 μL). When tumors reached a mean size of 200-250 mm³, animals were randomized into groups and administered saline vehicle (10 mL/kg, IP, days 1 and 2), filanesib (12.5 mg/kg, IP, days 1 and 2), pomalidomide (10 mg/kg, IP, QD, days 1-21 for RPMI-8226 and H929 or days 1-19 for JJN3), or the combination regimen of both compounds. Tumor size and animal body weight were measured on the days indicated in FIGS. 2-4 over the course of each study. Tumor volume was calculated using the formula, volume=(width²/length)/2. Percent complete response indicates an animal has no palapable tumor for 2 consecutive measurements. See FIGS. 2-4 and Table 2.

TABLE 2

| | % Complete Response | | | % Max Body Weight Loss | | |
|---|---|---|---|---|---|---|
| | RPMI-8226 | JJN3 | H929 | RPMI-8226 | JJN3 | H929 |
| Vehicle | 0 | 0 | 0 | 1.8 | 1.0 | 0.8 |
| filanesib | 50 | 0 | 0 | 5.3 | 6.6 | 8.9 |
| pomalidomide | 12.5 | 0 | 28.5 | 0.9 | 0 | 4.4 |
| combination | 100 | 100 | 57 | 7.4 | 7.9 | 11.6 |

### Example 3

### Hematology Profiles

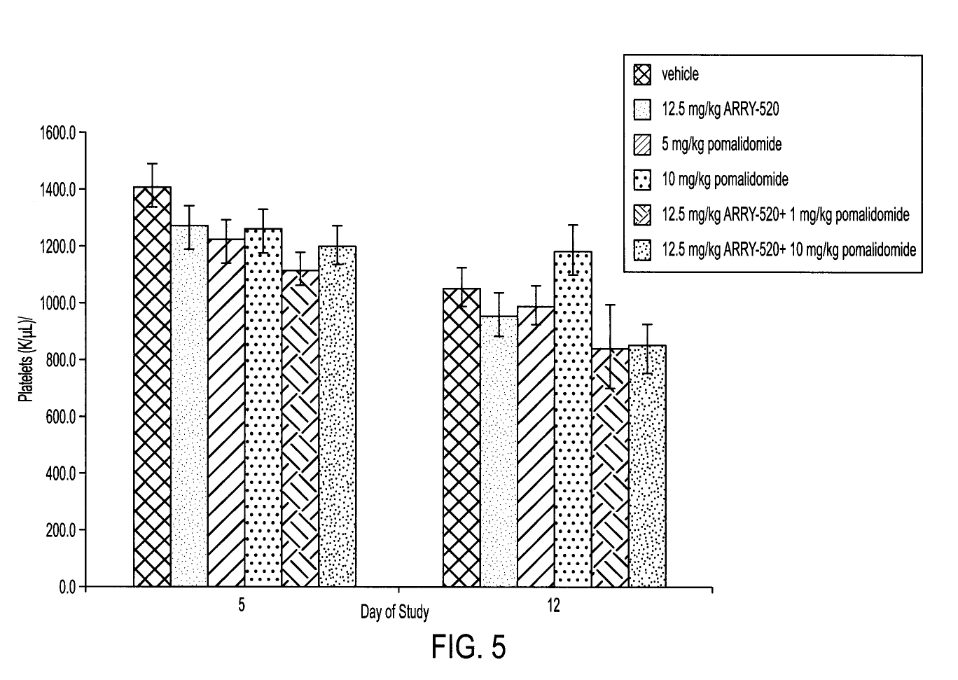

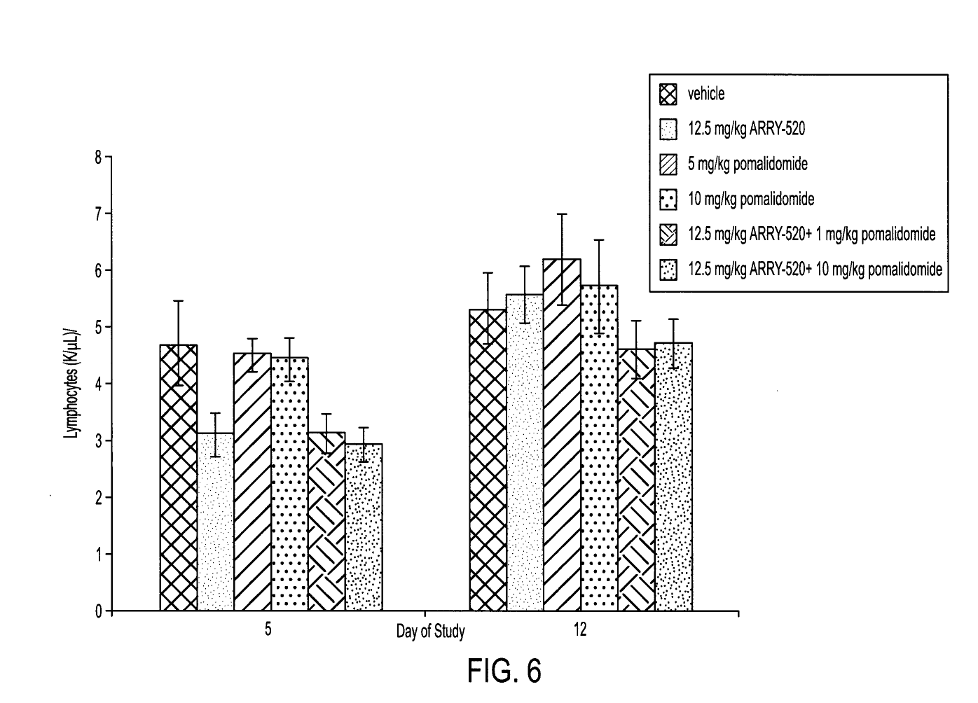

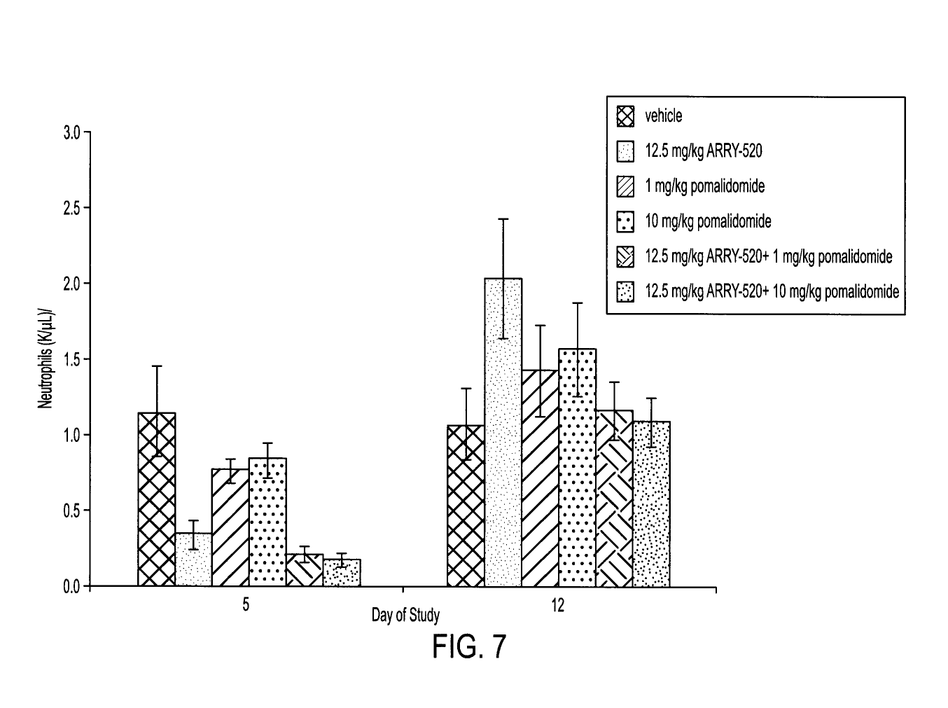

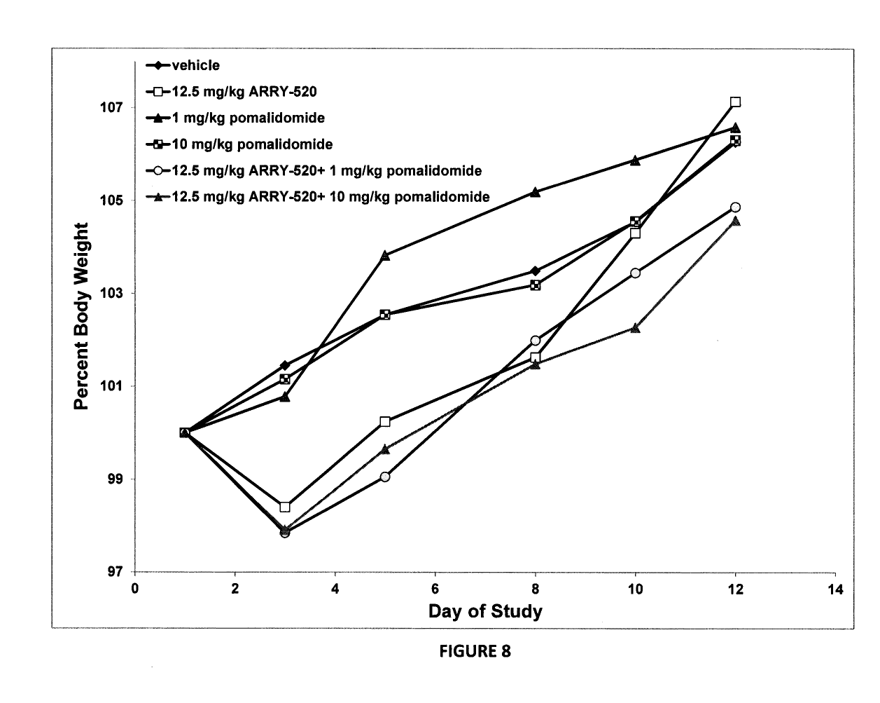

Naïve male CD-1 mice were administered saline vehicle (10 mL/kg, IP, days 1 and 2), filanesib (12.5 mg/kg, IP, days 1 and 2) and/or pomalidomide (1 or 10 mg/kg, IP, QD, days 1-14). Hematology parameters (platelets FIG. 5, lymphocytes FIG. 6, neutrophils FIG. 7) were measured on days 5 and 12, and animal body weight (FIG. 8) was measured on days 1, 3, 5, 8, 10 and 12. For hematology analysis, blood was collected 12 hours following the final dose via cardiac puncture, serum was prepared, and hematology profiles were measured on Hemavet 950FS analyzer. See FIGS. 5-8 and Table 3.

TABLE 3

| | % Decrease in Cell Counts from Vehicle | | | | | | |
|---|---|---|---|---|---|---|---|
| | Platelets | | Lymphocytes | | Neutrophils | | % Max Body |
| | Day 5 | Day 12 | Day 5 | Day 12 | Day 5 | Day 12 | Weight Loss |
| vehicle | — | — | — | — | — | — | 0 |
| 12.5 mg/kg filanesib | 9 | 0 | 34 | 0 | 69 | 0 | 0 |
| 1 mg/kg pomalidomide | 8 | 0 | 9 | 0 | 27 | 0 | 0 |
| 10 mg/kg pomalidomide | 10 | 0 | 4 | 0 | 21 | 0 | 0 |
| 12.5 mg/kg filanesib + | 21 | 17 | 33 | 11 | 86 | 0 | 2.2 |
| 1 mg/kg pomalidomide | | | | | | | |
| 12.5 mg/kg filanesib + | 17 | 16 | 37 | 10 | 88 | 0 | 2.1 |
| 10 mg/kg pomalidomide | | | | | | | |

### Example 4

### RPMI-8226 Dose Ranging Xenograft

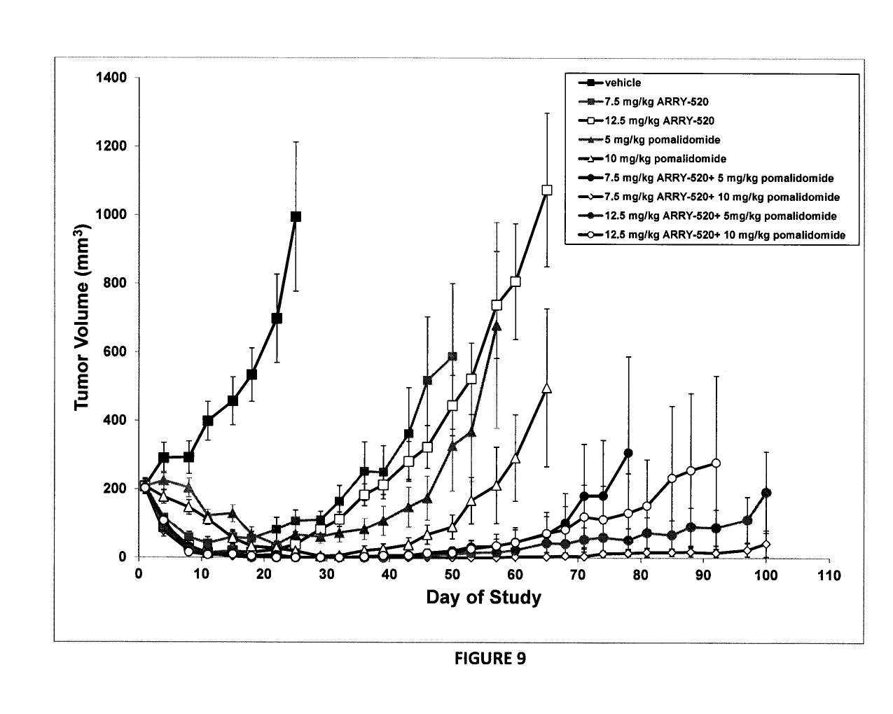

Female SCID-Beige mice were inoculated subcutaneously with 10×10⁶ RPMI-8226 tumor cells in 50% 1×PBS 50% Matrigel™ (100 μL). When tumors reached a mean size of 200 mm³, animals were randomized into groups and administered saline vehicle (10 mL/kg, IP, days 1 and 2), filanesib (7.5 or 12.5 mg/kg, IP, days 1 and 2), pomalidomide (5 or 10 mg/kg, IP, QD, days 1-21), or combinations of filanesib and pomalidomide. Tumor size and animal body weight were measured on the indicated days over the course of each study. Tumor volume was calculated using the formula, volume=(width²/length)/2. Percent cure rate indicates an animal has no palpable tumor 158 days from study start. See FIG. 9 and Table 4.

TABLE 4

| | | % Max Body |
|---|---|---|
| | % Cure Rate | Weight Loss |
| Vehicle | 0 | 1.6 |
| 7.5 mg/kg filanesib | 0 | 3.7 |
| 12.5 mg/kg filanesib | 0 | 8.0 |
| 5 mg/kg pomalidomide | 0 | 0.5 |
| 10 mg/kg pomalidomide | 0 | 1.4 |
| 7.5 mg/kg filanesib + 5 mg/kg | 62.5 | 3.4 |
| pomalidomide | | |
| 7.5 mg/kg filanesib + 10 mg/kg | 75 | 6.2 |
| pomalidomide | | |
| 12.5 mg/kg filanesib + 5 mg/kg | 50 | 7.7 |
| pomalidomide | | |
| 12.5 mg/kg filanesib + 10 mg/kg | 50 | 10.2 |
| pomalidomide | | |

It will be understood that the enumerated embodiments are not intended to limit the invention to those embodiments. On the contrary, the invention is intended to cover all alternatives, modifications and equivalents, which may be included within the scope of the present invention as defined by the claims. Thus, the foregoing description is considered as illustrative only of the principles of the invention.

The words “comprise,” “comprising,” “include,” “including,” and “includes” when used in this specification and in the following claims are intended to specify the presence of stated features, integers, components, or steps, but they do not preclude the presence or addition of one or more other features, integers, components, steps, or groups thereof.

1-12. (canceled)

13. A method of treating cancer comprising administering to a mammal in need of such treatment an effective amount of filanesib and pomalidomide.

14. The method of claim 13, wherein the cancer is selected from hematological tumors.

15. The method of claim 14, wherein the cancer is selected from lymphomas, leukemia and multiple myeloma.

16. The method of claim 13, wherein the cancer is selected from solid tumors.

17. The method of claim 16, wherein the cancer is selected from skin, breast, brain, cervical carcinoma, and testicular cancer.

18. The method of claim 16, wherein the cancer is selected from breast cancer, colorectal cancer, non-small cell lung cancer, pancreatic cancer, bladder cancer, salivary gland cancer (adenoid cystic), esophageal cancer, mesothelioma cancer, and mixed small cell lung cancer/non-small cell lung cancer.

19. A method of treating cancer in a mammal comprising administering a therapeutically effective amount of filanesib and pomalidomide to the mammal.

20. The method of claim 19, wherein the cancer is selected from hematological tumors.

21. The method of claim 20, wherein the cancer is selected from lymphomas, leukemia and multiple myeloma.

22. The method of claim 19, wherein the cancer is selected from solid tumors.

23. The method of claim 22, wherein the cancer is selected from skin, breast, brain, cervical carcinoma, and testicular cancer.

24. The method of claim 22, wherein the cancer is selected from breast cancer, colorectal cancer, non-small cell lung cancer, pancreatic cancer, bladder cancer, salivary gland cancer (adenoid cystic), esophageal cancer, mesothelioma cancer, and mixed small cell lung cancer/non-small cell lung cancer.
